(19) United States
(12) Patent Application Publication
(10) Pub. No.: US 2004/0170672 A1
(43) Pub. Date: Sep. 2, 2004

(54) Transdermal therapeutic system for administration of partial dopamine-d2 agonists

(76) Inventor: Thorsten Selzer, Neuwied (DE)

Correspondence Address: BIRCH STEWART KOLASCH & BIRCH, PO BOX 747, FALLS CHURCH, VA, 22040-0747, US

(21) Appl. No.: 10/469,917

(22) PCT Filed: Feb. 26, 2002
(86) PCT No.: PCT/EP02/02002

(30) Foreign Application Priority Data
Mar. 7, 2001 (DE) 10101953.9

(51) Int. Cl. A61K 9/70

(52) U.S. Cl. 424/449

(57) ABSTRACT

A transdermal therapeutic system (TTS) for administering at least one partial dopamine D2 agonist, comprising an active substance-impermeable backing layer, an active substance reservoir and a detachable protective layer, where the active substance reservoir is pressure-sensitive adhesive or the TTS is provided with at least one pressure-sensitive adhesive layer, and where the active substance reservoir is configured as a matrix system or as a membrane system, is characterized in that the active substance reservoir contains at least one active substance from the group of partial dopamine D2 agonists.

The present invention relates to transdermal therapeutic systems (TTS) for administering active agents from the group of partial dopamine D2 agonists to the skin of patients. It further relates to the use of such TTS for drug treatment of patients suffering from schizophrenic psychoses.

According to the theoretical model valid today, schizophrenic psychoses are considered to be clinical manifestations of an imbalance existing in the complicated network of neurotransmitters in the various regions of the brain. The neurotransmitter dopamine plays a central part as a modulator in this interplay.

Classic neuroleptics cause an almost complete, unspecific blockade of the dopamine D2 receptors. Their good efficacy on the productive positive symptoms has been proven, it is true, but these active substances simultaneously produce unacceptable extrapyramidal-motoric side effects. Therefore, the preferred aim is to bring about a normalisation —instead of a blockade—of the neuronal stimulus conduction. Dopamine activity is to be reduced in those regions of the brain where it is too strong, but not where it is normal. This requirement is met to a large extent by the active substance group of the partial D2 agonists.

Partial dopamine D2 agonists, which include, for example, the active substance aripiprazole, which belongs to the general substance class of the phenylpiperazinyl chinolinones, are characterized by the fact that they block the postsynaptic dopamine D2 receptors and at the same time stimulate the presynaptic autoreceptors. In this way excessive dopamine activity is suppressed, and the risk of extrapyramidal-motoric side effects is reduced at the same time.

The therapeutic efficacy and tolerance of aripiprazole was examined and confirmed in the USA in a randomized multicentric double-blind study in comparison to haloperidole and a placebo.

However, the partial dopamine D2 agonist aripiprazole, in particular, has some disadvantages which result form pharmacokinetics. Administered orally, aripiprazole is subject to intense metabolism during the first intestine-liver passage (first pass effect) and has a short plasma half-life. To maintain a therapeutically effective plasma level, a relatively high frequency of application is therefore necessary if aripiprazole is administered orally.

The task underlying the invention was therefore to provide the active substance aripiprazole (7-(4-(4-(2,3-dichlorophenyl)-1-piperazinyl)butoxy)-3,4-dihydro-2(1H)-chinolinone) or another active substance from the group of the partial D2 agonists in an administration form by means of which the aforementioned disadvantages can be avoided and which is advantageously suitable for therapeutic treatment of schizophrenic psychoses.

This task is solved by transdermal therapeutic systems (TTS) in plaster form according to claims 1 to 14. These systems enable an active substance flux in vivo sufficient for therapeutic purposes, and they can be produced by utilizing usual manufacturing processes.

The partial D2 agonists-containing TTS according to the invention after application release the active substance contained therein to the patient's skin, so that the active substance becomes systemically available. Due to the direct delivery of the active agent into the circulatory system, the rapid metabolism caused by the first pass effect as occurring in oral administration is avoided. In addition, the TTS according to the invention ensure a constant delivery of the partial D2 agonist(s) contained therein, during the period of application. In this manner, with a low application frequency it is possible to achieve that a relatively constant plasma level is maintained. The quick elimination of the partial dopamine D2 agonist (e.g. aripiprazole) is compensated by a continuous delivery of the active agent from the active agent reservoir of the TTS. In addition, due to the transdermal administration, problems such as e.g. gastrointestinal intolerance, low enteral absorption or low peroral availability are eluded.

The subject matter of the present invention is thus a transdermal therapeutic system (TTS) in patch form having the features mentioned in the preamble of claim 1, and whose active substance reservoir contains at least one active substance from the group of partial dopamine D2 agonists. Further preferred embodiments of the invention are described in the subclaims.

The structure of the TTS according to the invention comprises an active substance-impermeable backing layer, an active substance reservoir and a detachable protective layer. The active substance-containing reservoir itself may have pressure-sensitive adhesive properties, or a pressure-sensitive adhesive layer may be provided which enables fixation of the TTS on the skin. The backing layer connected with the active substance reservoir covers the TTS on the side averted from the skin. The detachable and active substance-insoluble protective layer during storage covers the pressure-sensitive adhesive TTS-surface facing the skin and is detached before application.

The invention comprises both TTS configured as matrix systems and TTS configured as membrane systems.

The TTS according to the invention can be used both in the form of matrix systems as well as in the form of pouch or membrane systems. In the case of a matrix system, the active substance matrix can for example be a plastic or synthetic resin matrix serving as active substance reservoir and containing the active substance in dissolved or dispersed form. It is preferably pressure-sensitive adhesive and can be embodied so as to be both single- and two- or multi-layered. The term “matrix systems” also includes such embodiments where the active substance reservoir is a fibre material, for example a cotton woven fabric or cotton nonwoven, to which the active substance, e.g. aripiprazole, is adsorbed. This fibre material may be embedded in a plastic matrix or synthetic resin matrix.

In principle, a plurality of polymers, resins and additives known to those skilled in the art can be taken into consideration for the production of active substance-containing additives; however, care must be taken that these substances —in so far as coming into contact with the skin—are tolerated by the skin, and that the formulation is suitable for delivering the active substance aripiprazole or another dopamine D2 agonist to the skin.

Suitable base polymers for producing the active substance matrix or the pressure-sensitive adhesive layer of the TTS according to the invention are polymers based on acrylic acid and its esters, isobutylene, ethylene-vinyl acetate copolymers, natural rubbers, synthetic rubbers such as styrene-diene copolymers, especially styrene-butadiene block copolymers, isoprene block polymers, acrylonitrile-butadiene rubber, butyl rubber or neoprene rubber, as well as pressure-sensitive adhesives based on silicone, or “hot-melt adhesives”. The term “hot-melt adhesives” comprises any adhesives which are not liquefied with solvents but by melting at elevated temperature, preferably in the range of from 60-200° C. Suitable as hot-melt adhesives are, in particular, mixtures of esters of hydrogenated colophony with cellulose derivatives. The mentioned base polymers may also be used in form of suitable mixtures.

Apart from the above-mentioned polymers other polymers known to the skilled artisan may also be used as base polymers for producing the matrix or the pressure-sensitive adhesive layer, provided they are compatible with the active substance aripiprazole or the respective partial dopamine D2 agonists utilized.

The active substance aripiprazole, or another partial dopamine D2 agonist, is in the simplest case dispersed, coarsely, colloidally or molecularly, in a solution or melt of base polymers. The further manufacture of the TTS can take place in such a manner that this active substance-containing mixture is coated onto a suitable support, for example to a thermoplastic film provided with a silicone layer, and—possibly after evaporation of the solvent components —is covered with a further film which will later constitute the backing layer of the TTS. By punching flat-shaped objects of the desired geometric shape, TTS are made from such a laminate. The pharmaceutically acceptable substances suitable as auxiliaries such as plasticizers, tackifiers, solubilizers, stabilizers, fillers, carrier substances and permeation enhancers are in principle known to those skilled in the art.

According to a further embodiment it is provided for the active substance to be present in a bag- or pouch-shaped reservoir of the TTS according to the invention. The reservoir is filled with a flowable, e.g. viscous or high-viscous or semi-solid, plastic matrix, or with a solution thereof, containing the active substance. It is of particular advantage if the active substance reservoir contains a gel former. The rear side of the pouch, which is averted from the skin, in this case must be active substance-impermeable, whereas the side of the pouch facing the skin must be permeable to active substance. To control the active substance delivery, an active substance-permeable membrane may be arranged on the side facing the skin (“membrane system”). Suitable materials for producing such a pouch and suitable materials for the membrane, as well as suitable gel formers, are known to those skilled in the art.

A preferred embodiment of the invention consists in that the active substance aripiprazole, or another active substance from the group of partial dopamine D2 agonists is present in the reservoir of the TTS in dissolved condition; in this case the formulation should, if possible, contain a solubilizer. Preferred examples for solubilizers are polyhydric alcohols, especially 1,2-propanediol, butanediol, glycerine, polyethylene glycol 400, tetrahydrofurfuryl alcohol, diethyleneglycol monoether, diethyl toluamide and monoisopropylidene glycerine; 1,2-propanediol is used with particular preference. It has proved to be of advantage for the portion of the solvent to be 1 to 50%-wt, especially preferred 5 to 35%-wt, relative to the overall weight of the active substance reservoir. It is to be taken into consideration that some of the mentioned solubilizers, e.g. 1,2-propanediol, can simultaneously act as permeation enhancing substances.

To obtain a high active substance flux through the skin, it has proved particularly advantageous, especially in matrix systems, for the active substance-containing matrix to contain permeation-enhancing substances in an amount of 0.1 to 25%-wt, preferably from 1 to 10%-wt, in each case relative to the total weight of the active substance matrix. Preferred examples for skin permeation-enhancing additives are fatty alcohols such as decanols and dodecanols, as well as fatty acids such as oleic acid or myristic acid, as well as polyoxethylene fatty alcohol ethers, preferably polyoxylauryl ether (e.g. Brij®), as well as polyoxyethylene fatty acid esters, fatty acid esters of sorbitane monolaurate, esters of long-chain fatty acids with methyl, ethyl or isopropyl alcohol, esters of fatty alcohols with acetic acid or lactic acid, as well as oleic acid diethanolamine. The permeation-enhancing substances mentioned may be added either singly or as a mixture.

To achieve a high active substance release rate, the active substance concentration in the active substance matrix or the active substance-containing layers is preferably as high as possible. In this connection it has to be borne in mind, however, that the physical stability of the active substance may be adversely affected if the concentrations are too high. In the inventive TTS, therefore, active substance concentrations are employed which are in the range 0.1 to 50%-wt, in particular from 1 to 10%-wt, in each case relative to the total mass of the active substance reservoir.

According to the invention, it is further provided that the active substance aripiprazole, or another partial dopamine D2 agonist, be present in combination with at least one further active substance. With preference, this active substance or these active substances are from the substance class of the phenothiazines and/or their analogues, and/or from the class of butyrophenones, and/or from the class of diphenylbutyl piperidines.

Furthermore the active substance matrix or individual layers of the matrix may contain plasticizers which are in principle known to those skilled in the art. The concentration of these plasticizers may amount to up to 30%-wt, and is preferably between 5 and 20%-wt, in each case relative to the active substance matrix. The plasticizers may be selected, for example, from the groups of the hydrocarbons, alcohols, carboxylic acids and their derivatives, ethers, esters or amines.

To enable a control of the active substance release, unless accomplished by other mechanisms, the active substance reservoir may also be provided, on the side which is near the skin and releases active agent, with a control membrane controlling the release of active substance to the skin (“membrane system”).

The invention also encompasses such embodiments where the active substance matrix has a two- or multi-layered structure. For instance, the various matrix layers may contain polymer constituents from the group of substituted celluloses, preferably of the methyl and ethyl celluloses. In this case, the matrix layers may be configured such that they differ from each other in terms of their polymer or pressure-sensitive adhesive composition, their active substance concentration, their concentration of permeation-enhancing additives or of solubilizers. In accordance with the intended application purpose, the above-mentioned concentrations can be made to differ such that the concentrations become smaller or greater from the side which is away from the skin in the direction toward the matrix layer which is in proximity to the skin, depending on whether a special long-term action or an initial effect is aimed at.

Fixation of the TTS according to the invention may be achieved in various ways. For example, there is a possibility for the active substance matrix itself to be made of a pressure-sensitive adhesive or of a mixture of pressure-sensitive adhesive polymers. Fixation of the TTS on the skin by means of an additional, active substance-free pressure-sensitive adhesive layer is also possible. Moreover, it is also conceivable that in such inventive TTS as are provided with a control membrane, fixation on the skin is achieved by means of a margin of adhesive. This margin of adhesive does not touch the active substance-releasing surface, and is thus not connected with the control membrane.

The TTS according to the present invention, apart from their active substance reservoir, have an active substance impermeable backing layer as well as a likewise active substance-impermeable protective layer or stripping film. Suitable as materials for the backing layer are, above all, polyesters which are characterized by a particular strength, e.g. polyethylene terephthalate and polybutylene terephthalate, but apart from these also any other skin-tolerated plastics such as polyvinyl chloride, ethylenevinyl acetate copolymers, polyvinyl acetate, polyethylene, polypropylene, polyurethane, cellulose derivatives and many more. In the individual case, the backing layer may be provided with an additional layer, e.g. by vapour deposition of metals, especially aluminium.

For the detachable protective layer basically the same materials may be used as for the backing layer, provided that they are made detachable by a suitable surface treatment, e.g. siliconization. However, other detachable protective layers such as polyetrafluoroethylene-treated paper or cellophane® (cellulose hydrate) may also be utilized.

The TTS according to the invention are advantageously suitable for acute and long-term therapy of schizophrenic psychoses. Here, the TTS containing the active substance aripiprazole or another partially dopamine D2 agonist is applied to the skin of such a patient and is left there for a period of at least 8 hours. The application period may last up to three days.

The TTS according to the invention can be prepared, for example, as follows:

### EXAMPLE

50 g of the active substance aripiprazole and 20 g of a suitable permeation-enhancing substance (e.g. Brij® 30) are dissolved in 200 g of 1,2-propanediol. This solution is introduced as base polymer in a silicone adhesive (No. 4301, by the firm of Dow Corning, USA) by means of a suitable stirring apparatus, so that a liquid-liquid dispersion results that is as homogenous as possible. This dispersion is uniformly coated on a carrier film (e.g. of polyethylene terephthalate) using an appropriate device. Subsequently the solvent of the silicone adhesive as well as possible portions of propanediols are removed by controlled drying. The laminate thus obtained is subsequently laminated with a further film of polyethylene terephthalate. Finally, TTS of a pre-determined area are punched out and packed in suitable packages.

1. Transdermal therapeutic system (TTS) for administering at least one partial dopamine D2 agonist, having an active substance-impermeable backing layer, an active substance reservoir and a detachable backing layer, where the active substance reservoir is pressure-sensitive adhesive or the TTS has at least one pressure-sensitive adhesive layer, and where the active substance reservoir is configured as a matrix system or as a membrane system, characterized in that the active substance reservoir contains at least one active substance from the group of the partial dopamine D2 agonists.

2. TTS according to claim 1, characterized in that the active substance reservoir is configured as a single-, double- or multilayered active substance matrix.

3. TTS according to claim 2, characterized in that the active substance matrix is a plastics or synthetic resin matrix, preferably a pressure-sensitive adhesive matrix, where the basic polymer(s) of this matrix is/are preferably selected from the group comprising polymers based on acrylic acid and its esters, isobutylenes, ethylene-vinyl acetate copolymers, natural rubbers, synthetic rubbers such as styrene-diene copolymers, especially styrene-butadiene block copolymers, isoprene block polymers, acrylonitrile-butadiene rubber, butyl rubber and neoprene rubber, as well as pressure-sensitive adhesives based on silicone, as well as hot-melt adhesives, preferably mixtures of esters of hydrogenated colophony with cellulose derivatives.

4. TTS according to any one of the preceding claims, characterized in that the active substance reservoir contains a fibre material, a woven fabric or a nonwoven, to which the active substance is adsorbed.

5. TTS according to claim 1, characterized in that the active substance reservoir is configured as a pouch-shaped reservoir which contains the active substance in a flowable, viscous, semi-solid, gel-like or liquid preparation or solution and is confined on the side facing the skin by an active substance-permeable layer and on the side averted from the skin by an active substance-impermeable layer.

6. TTS according to any one of the preceding claims, characterized in that it additionally has an active substance-permeable membrane which modifies or controls the rate of active substance release.

7. TTS according to any one of the preceding claims, characterized in that aripiprazole is contained in a concentration in the range of from 0.1 to 50%-wt., preferably from 1 to 10%-wt, in each case relative to the total mass of the active substance reservoir.

8. TTS according to any one of the preceding claims, characterized in that aripiprazole is present in the active substance reservoir in dissolved state.

9. TTS according to any one of the preceding claims, characterized in that the active substance reservoir contains at least one solubilizer, preferably in an amount of from 1 to 50%-wt., with particular preference from 5 to 35%-wt., in each case relative to the total weight of the active substance reservoir.

10. TTS according to claim 9, characterized in that the solubilizers(s) is/are selected from the group comprising polyhydric alcohols, especially 1,2-propanediol, butanediol, glycerine, polyethylene glycol 400, tetrahydrofurfuryl alcohol, diethyleneglycol monoether, diethyltoluamide and monoisopropylidene glycerine.

11. TTS according to any one of the preceding claims, characterized in that the active substance reservoir contains at least one permeation-enhancing substance, preferably in an amount of from 0.1 to 25%-wt, with particular preference from 1 to 10%-wt, in each case relative to the total weight of the active substance reservoir.

12. TTS according to claim 8, characterized in that the permeation-enhancing substance(s) is/are selected from the group comprising fatty alcohols, preferably decanol and dodecanol, as well as fatty acids, preferably oleic acid, myristic acid, as well as polyoxyethylene fatty alcohol ethers, preferably polyoxylauryl ether, as well as polyoxyethylene fatty acid esters, fatty acid esters of sorbitane monolaurate, esters of long-chain fatty acids with methyl, ethyl or isopropyl alcohol, esters of fatty alcohols with acetic acid or lactic acid, as well as oleic acid diethanolamine.

13. TTS according to any one of the preceding claims, characterized in that it additionally contains at least one active substance selected from the group comprising phenothiazines and its analogues, butyrophenones and diphenylbutyl piperidines.

14. The use of aripiprazole for production of a transdermal therapeutic system comprising an active substance-impermeable backing layer, an active substance reservoir and a detachable protective layer, where the active substance reservoir is pressure-sensitive adhesive or the TTS has at least one pressure-sensitive adhesive layer.

15. Use of an aripiprazole-containing TTS according to any one of claims 1 to 13 for treating acute and/or chronic symptoms of schizophrenic psychoses.

16. Method of drug treatment of acute and/or chronic symptoms of schizophrenic psychoses, characterized in that a TTS containing aripiprazole is applied to the skin of a patient suffering from a schizophrenic psychosis.

17. Method according to claim 16, characterized in that the said TTS is a TTS according to any one of claims 1-13.

18. Method according to claim 16 or 17, characterized in that the application period of the TTS is at least 8 hours and maximally 3 days.

19. Method for administering aripiprazole via the skin to a person in need of a partial dopamine D2 agonist, characterized in that
a) a transdermal therapeutic system (TTS), comprising an active substance-impermeable backing layer, an active substance reservoir containing aripiprazole, a pressure-sensitive adhesive layer, possibly an active substance-permeable membrane, and a detachable protective layer, is freed from the detachable protective layer,
b) the TTS is applied with its pressure-sensitive adhesive layer to the intact skin of the patient,
c) the aripiprazole contained in the active substance reservoir is released from the active substance reservoir through the pressure-sensitive adhesive layer and possibly the active substance-permeable membrane over a period of at least 8 hours to the patient's skin, and
d) the TTS is removed from the patient's skin after a period of 8 hours, maximally, however, after a period of 3 days, from the patient's skin.

20. Process according to claim 19, characterized in that the person in need of a partial dopamine D2 agonist is a patient suffering from acute or chronic symptoms of schizophrenic psychoses.
